(19) United States
(12) Patent Application Publication
(10) Pub. No.: US 2019/0231991 A1
(43) Pub. Date: Aug. 1, 2019

(54) NEBULIZER ASSEMBLY AND AUXILIARY FLOW-GUIDING ELEMENT THEREOF

(71) Applicant: HCMed Innovations Co., LTD., Taipei City (TW)

(72) Inventors: Wen-Yu Tsai, Taipei City (TW); Ke-Ting Chen, Taipei City (TW); Chieh-Sheng Cheng, Taipei City (TW)

(21) Appl. No.: 16/199,247

(22) Filed: Nov. 26, 2018

(30) Foreign Application Priority Data
Jan. 31, 2018 (TW) 107103456

(51) Int. Cl. A61M 11/00; A61M 15/00

(52) U.S. Cl. CPC A61M 11/002; A61M 15/0021; A61M 11/005; A61M 15/002

(57) ABSTRACT

A nebulizer assembly includes a nebulizer host and an auxiliary flow-guiding element. The auxiliary flow-guiding element is disposed on the nebulizer host. The auxiliary flow-guiding element has an auxiliary flow-guiding channel and at least one auxiliary bottom flow-guiding opening communicating with the auxiliary flow-guiding channel, and the auxiliary bottom flow-guiding opening penetrates through a bottom portion of the auxiliary flow-guiding element. A nebulized fluid generated by the nebulizer host can be sent to the auxiliary flow-guiding channel of the auxiliary flow-guiding element, and an outside air can pass through the auxiliary bottom flow-guiding opening and flow into the auxiliary flow-guiding channel. Therefore, the nebulized fluid in the auxiliary flow-guiding channel can be propelled towards a direction away from the auxiliary bottom flow-guiding opening by the outside air flowing into the auxiliary flow-guiding channel through the auxiliary bottom flow-guiding opening.

### CROSS-REFERENCE TO RELATED PATENT APPLICATION

This application claims the benefit of priority to Taiwan Patent Application No. 107103456, filed on Jan. 31, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizer assembly and an auxiliary flow-guiding element thereof, and more particularly to a nebulizer assembly with an auxiliary bottom flow-guiding opening and an auxiliary flow-guiding element thereof.

### BACKGROUND OF THE DISCLOSURE

Nebulization therapies for respiratory diseases have gained their popularity in recent years for being capable of providing more efficient and more direct therapeutic effects than oral medication. Specifically, nebulized particles nebulized by a nebulizer can be fluid droplets at sizes of 3˜5 μm (for example, therapeutic droplets), and therefore can be inhaled from mouth and nose into the bronchus and then into the entire alveoli for treatment, so that the therapeutic droplets can be thoroughly absorbed by the body. At present, nebulizing methods employed by medical nebulizer products can be pneumatic or ultrasonic (such as a piezoelectric nozzle). However, there is still room for improvement for the conventional nebulizer.

### SUMMARY OF THE DISCLOSURE

In response to the above-referenced technical inadequacies, the present disclosure provides a nebulizer host and its auxiliary flow-guiding element.

In certain aspects, the present disclosure provides a nebulizer assembly including a nebulizer host, a main flow-guiding element and an auxiliary flow-guiding element. The nebulizer host includes a casing module and a nebulizing module disposed in the casing module. The main flow-guiding element is disposed on the casing module and corresponds to the nebulizing module. The auxiliary flow-guiding element is disposed on the main flow-guiding element. The casing module includes a communicating tube having an outlet channel, and the nebulizing module includes an outlet that communicates with the outlet channel. The auxiliary flow-guiding element has an auxiliary flow-guiding channel and at least one auxiliary bottom flow-guiding opening communicating with the auxiliary flow-guiding channel. The at least one auxiliary bottom flow-guiding opening penetrates a bottom portion of the auxiliary flow-guiding element. A nebulized fluid generated by the nebulizing module of the nebulizer host is sent to the auxiliary flow-guiding channel of the auxiliary flow-guiding element through the guiding of the main flow-guiding element, an outside air is introduced into the auxiliary flow-guiding channel by passing through the at least one auxiliary bottom flow-guiding opening, and the nebulized fluid in the auxiliary flow-guiding channel is propelled towards a direction away from the at least one auxiliary bottom flow-guiding opening by the outside air flowing into the auxiliary flow-guiding channel through the at least one auxiliary bottom flow-guiding opening.

In certain aspects, the present disclosure provides a nebulizer assembly including a nebulizer host, a main flow-guiding element and an auxiliary flow-guiding element. The main flow-guiding element is disposed on the casing module. The auxiliary flow-guiding element is disposed on the main flow-guiding element. The auxiliary flow-guiding element has an auxiliary flow-guiding channel and at least one auxiliary bottom flow-guiding opening communicating with the auxiliary flow-guiding channel. The at least one auxiliary bottom flow-guiding opening penetrates a bottom portion of the auxiliary flow-guiding element. A nebulized fluid generated by the nebulizing module of the nebulizer host is sent to the auxiliary flow-guiding channel of the auxiliary flow-guiding element through the guiding of the main flow-guiding element, an outside air is introduced into the auxiliary flow-guiding channel by passing through the at least one auxiliary bottom flow-guiding opening, and the nebulized fluid in the auxiliary flow-guiding channel is propelled towards a direction away from the at least one auxiliary bottom flow-guiding opening by the outside air flowing into the auxiliary flow-guiding channel through the at least one auxiliary bottom flow-guiding opening.

In certain aspects, the present disclosure provides an auxiliary flow-guiding element. The auxiliary flow-guiding element is disposed on a nebulizer host. The auxiliary flow-guiding element has an auxiliary flow-guiding channel and at least one auxiliary bottom flow-guiding opening communicating with the auxiliary flow-guiding channel. The at least one auxiliary bottom flow-guiding opening penetrates a bottom portion of the auxiliary flow-guiding element. A nebulized fluid generated by the nebulizing module of the nebulizer host is sent to the auxiliary flow-guiding channel of the auxiliary flow-guiding element, and an outside air is introduced into the auxiliary flow-guiding channel by passing through the at least one auxiliary bottom flow-guiding opening.

One of the beneficial effects of the present disclosure lies in that, through technical features of the auxiliary flow-guiding element having an auxiliary flow-guiding channel and at least one auxiliary bottom flow-guiding opening, and that of the at least one auxiliary bottom flow-guiding opening penetrating a bottom portion of the auxiliary flow-guiding element, a nebulized fluid generated by the nebulizer host can be sent into the auxiliary flow-guiding channel of the auxiliary flow-guiding element, and an outside air can be introduced into the auxiliary flow-guiding channel through the auxiliary bottom flow-guiding opening. As a result, the nebulized fluid in the auxiliary flow-guiding channel can be propelled towards a direction away from the at least one auxiliary bottom flow-guiding opening by the introduction of the outside air.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the detailed description and the accompanying drawings, in which:

FIG. 1 is an exploded view of a nebulizer assembly according to a first embodiment of the present disclosure.

FIG. 2 is a schematic view of a nebulizer assembly according to a first embodiment of the present disclosure.

FIG. 3 is a cross-sectional view taken along line of FIG. 2.

FIG. 4 is a cross-sectional view taken along line IV-IV of FIG. 2.

FIG. 5 is a cross-sectional view of another embodiment of a main flow-guiding element of the present disclosure.

FIG. 6 is an exploded view of a nebulizer assembly according to a second embodiment of the present disclosure.

FIG. 7 is a schematic view of a nebulizer assembly according to a second embodiment of the present disclosure.

FIG. 8 is a cross-sectional view taken along line VIII-VIII of FIG. 7.

FIG. 9 is a cross-sectional view taken along line IX-IX of FIG. 7.

FIG. 10 is a cross-sectional view of another embodiment of a main flow-guiding element of the present disclosure.

FIG. 11 is an exploded view of a nebulizer assembly according to a third embodiment of the present disclosure.

FIG. 12 is a schematic view of an auxiliary flow-guiding element according to a third embodiment of the present disclosure.

FIG. 13 is a schematic view of a nebulizer assembly according to a third embodiment of the present disclosure.

FIG. 14 is a cross-sectional view taken along line XIV-XIV of FIG. 13.

FIG. 15 is a partial cross-sectional view of a nebulizer assembly according to a fourth embodiment of the present disclosure.

FIG. 16 is a partial cross-sectional view of a nebulizer assembly according to a fifth embodiment of the present disclosure.

FIG. 17 is a partial cross-sectional view of a nebulizer assembly according to a sixth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Various embodiments of the disclosure are now described in detail. Referring to the drawings, like numbers, if any, indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present disclosure. Additionally, some terms used in this specification are more specifically defined below.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be expressed in more than one way. Consequently, alternative language and synonyms may be used for any one or more of the terms discussed herein, and no special significance is to be placed upon whether or not a term is elaborated or discussed herein. Synonyms for certain terms may be provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including any definitions given herein, will prevail.

While numbering terms such as “first”, “second” or “third” may be used in this disclosure to describe various components, signals or the like, the terms are for distinguishing one component from another component, or one signal from another signal only, and are not intended to, nor should they be construed to impose any other substantive descriptive limitations on the components, signals or the like.

### First Embodiment

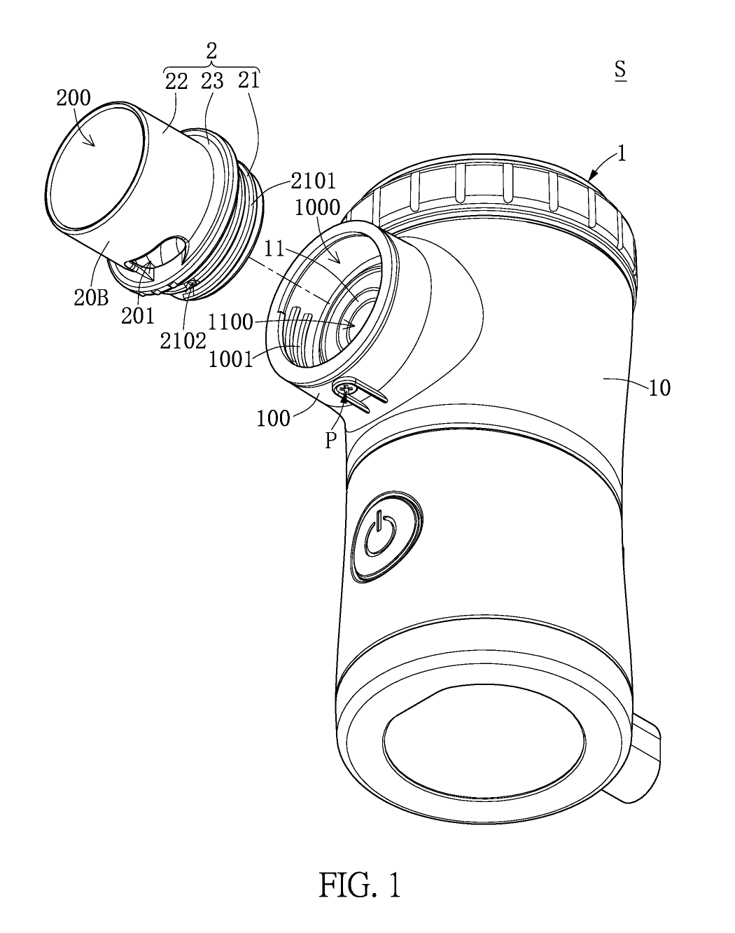

Referring to FIG. 1 to FIG. 4, a first embodiment of the present disclosure provides a nebulizer assembly S including a nebulizer host 1 and a main flow-guiding element 2 (or, namely, a “first flow-guiding element,” that is, the term “main” is interchangeable with the term “first” regarding the main flow-guiding element throughout this disclosure).

First, as shown in FIG. 1 to FIG. 3, the nebulizer host 1 includes a casing module 10 and a nebulizing module 11 disposed in the casing module 10. In certain embodiments, the casing module 10 includes a communicating tube 100 having an outlet channel 1000, and the nebulizing module 11 includes an outlet 1100 (i.e., an outlet for nebulized fluid) that communicates with the outlet channel 1000. The nebulizing module 11 is configured to generate a nebulized fluid F and send the nebulized fluid F out of the outlet 1100, and then, in certain embodiments, to a main flow-guiding channel 200 of a main flow-guiding element 2 and/or to an auxiliary flow-guiding channel 300 of an auxiliary flow-guiding element 3. In certain embodiments, the nebulizing module 11 may include a nebulizing sheet having a plurality of nebulizing holes and a vibrating sheet that operates in coordination with the nebulizing sheet. However, the present disclosure is not limited to the above-mentioned examples. Rather, the nebulizing module 11 of the present disclosure directs to and encompasses any module or structure capable of nebulizing fluid.

Further, as shown in FIG. 1 to FIG. 3, the main flow-guiding element 2 is disposed on the casing module 10 and corresponds to the nebulizing module 11. Furthermore, the main flow-guiding element 2 has a main flow-guiding channel 200 and at least one main bottom flow-guiding opening 201 communicating with the main flow-guiding channel 200. The at least one main bottom flow-guiding opening 201 penetrates a bottom portion 20B of the main flow-guiding element 2. In certain embodiments, the main flow-guiding element 2 may be a spray nozzle. The main flow-guiding element 2 has a first matching portion 21, a second matching portion 22, and a limiting portion 23 connected between the first matching portion 21 and the second matching portion 22. In addition, the main flow-guiding element 2 can be fixed in position on the casing module 10 through the matching of the first matching portion 21 and the communicating tube 100, and the limiting portion 23 can abut against the outer surface of the communicating tube 100. In addition, the outer surface of the first matching portion 21 has external thread 2101 and a positioning hole 2102, and the inner surface of the communication tube 100 has internal thread 1001 matching with the external thread 2101. In addition, a fixing element P can penetrate through the communication tube 100 and be connected between the communication tube 100 and the positioning hole 2102, and the main bottom flow-guiding opening 201 is provided on the second matching portion 22. That is, when the first matching portion 21 is inserted into the communication tube 100 and the limiting portion 23 abuts against the outer surface of the communication tube 100, the main flow-guiding element 2 can be fixed in position on the casing module 10 through the matching of the external thread 2101 and the internal thread 1001. However, the present disclosure is not limited thereto.

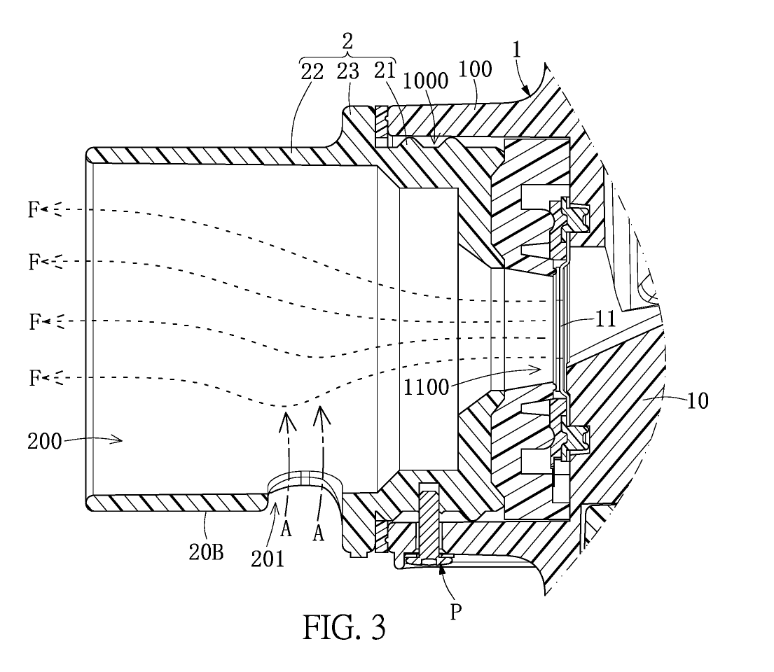

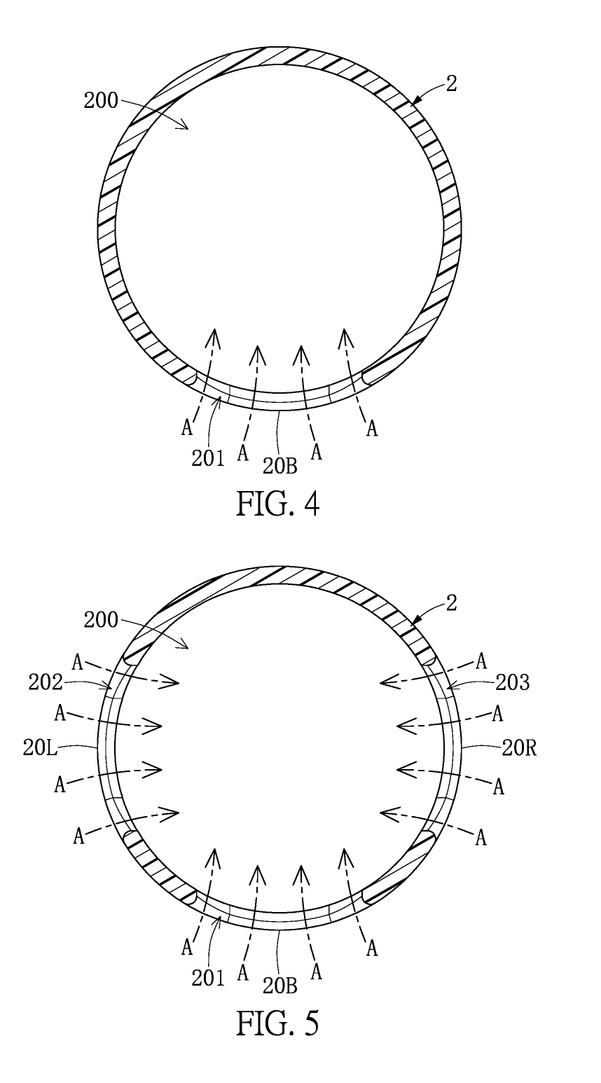

Further, as shown in FIG. 3 and FIG. 4, when the nebulizer host 1 is turned on, a nebulized fluid F generated by the nebulizing module 11 of the nebulizer host 1 can be sent to the main flow-guiding channel 200 of the main flow-guiding element 2 (for example, the nebulized fluid F can be directly sent to the main flow-guiding channel 200, or the nebulized fluid F can be sent to the main flow-guiding channel 200 through the communication tube 100), and outside air A can be introduced into the main flow-guiding channel 200 through the main bottom flow-guiding opening 201. As a result, as shown in FIG. 3, the nebulized fluid F in the main flow-guiding channel 200 can be propelled towards a direction away from the at least one main bottom flow-guiding opening 201 by the introduction of the outside air A from the main bottom flow-guiding opening 201. The nebulized fluid F in the main flow-guiding channel 200 can also be propelled along a direction from the outlet 1100 to the main flow-guiding channel 200 by the introduction of the outside air A from the main bottom flow-guiding opening 201. That is, when the nebulized fluid F generated by the nebulizing module 11 is sent into the main flow-guiding channel 200 of the main flow-guiding element 2, a negative pressure is formed in the main flow-guiding channel 200, enabling the outside air A to be introduced into the main flow-guiding channel 200 through the main bottom flow-guiding opening 201. Therefore, the nebulized fluid F in the main flow-guiding channel 200 can be propelled towards a direction away from the at least one main bottom flow-guiding opening 201 and propelled along a direction from the outlet 1100 to the main flow-guiding channel 200 by the introduction of the outside air A from the main bottom flow-guiding opening 201.

For example, when the nebulized fluid F carries a plurality of therapeutic particles, since the nebulized fluid F in the main flow-guiding channel 200 can be propelled towards a direction away from the at least one main bottom flow-guiding opening 201 by the introduction of the outside air A, the nebulized fluid F carrying the therapeutic particles will not easily fall (attach) onto the inner surface of the main flow-guiding element 2, so that medicine waste can be reduced. In this way, by employing the main flow-guiding element 2 with the main bottom flow-guiding opening 201, the present disclosure enables the therapeutic particles carried by the nebulized fluid F to be smoothly carried to the outside of the main flow-guiding element 2, so as to provide a sufficiently effective dosage of medicine for a user to inhale (that is, improving the effectiveness of medicating). However, the present disclosure is not limited to this example.

In summary, with reference to FIG. 1 to FIG. 3, a first embodiment of the present disclosure provides a nebulizer assembly S including a nebulizer host 1 and a main flow-guiding element 2, and the main flow-guiding element 2 is disposed on the nebulizer host 1. The main flow-guiding element 2 has a main flow-guiding channel 200 and at least one main bottom flow-guiding opening 201 communicating with the main flow-guiding channel 200. The main bottom flow-guiding opening 201 penetrates a bottom portion 20B of the main flow-guiding element 2. A nebulized fluid F generated by the nebulizer host 1 can be sent to the main flow-guiding channel 200 of the main flow-guiding element 2, and an outside air A can be introduced through the main bottom flow-guiding opening 201 into the main flow-guiding channel 200.

It is worth noting that, in certain embodiments, as shown in FIG. 5, the main flow-guiding element 2 further has at least one main left flow-guiding opening 202 communicating with the main flow-guiding channel 200, and at least one main right flow-guiding opening 203 communicating with the main flow-guiding channel 200. In certain embodiments, the main left flow-guiding opening 202 penetrates a left end portion 20L of the main flow-guiding element 2, and the main right flow-guiding opening 203 penetrates a right end portion 20R of the main flow-guiding element 2. In this way, the outside air A can be introduced into the main flow-guiding channel 200 through the main left flow-guiding opening 202 and the main right flow-guiding opening 203. In certain embodiments, when the nebulized fluid F generated by the nebulizing module 11 is sent to the main flow-guiding channel 200 of the main flow-guiding element 2, the outside air A is introduced into the main flow-guiding channel 200 by passing through the at least one main left flow-guiding opening 202 and the at least one main right flow-guiding opening 203. When the outside air A is introduced into the main flow-guiding channel 200 through the main left flow-guiding opening 202 and the main right flow-guiding opening 203, the nebulized fluid F inside the main flow-guiding channel 200 (as shown in FIG. 3) can be propelled along a direction from the outlet 1100 to the main flow-guiding channel 200 by the introduction of the outside air A through the main left flow-guiding opening 202 and the main right flow-guiding opening 203. In certain embodiments, the main left flow-guiding opening 202 and the main right flow-guiding opening 203 are relatively close to the main bottom flow-guiding opening 201. For example, at least one of the centers of the main left flow-guiding opening 202 and the main right flow-guiding opening 203 is closer to a center of the main bottom flow-guiding opening 201 than to a center of a vertical projection of the main bottom flow-guiding opening 201 projected along an axis perpendicular to a plane where the main bottom flow-guiding opening 201 is located and projected on a side of the main flow-guiding element 2 opposite to the main bottom flow-guiding opening 201, allowing the nebulized fluid F in the main flow-guiding channel 200 to be propelled towards a direction away from the at least one main bottom flow-guiding opening 201 to a greater extent by the introduction of the outside air A through the main left flow-guiding opening 202 and the main right flow-guiding opening 203.

### Second Embodiment

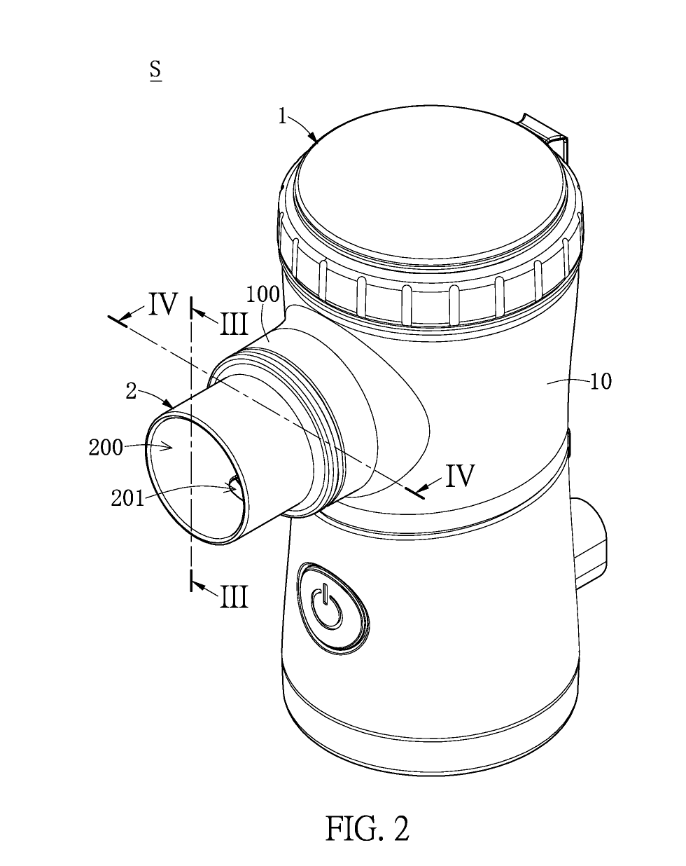

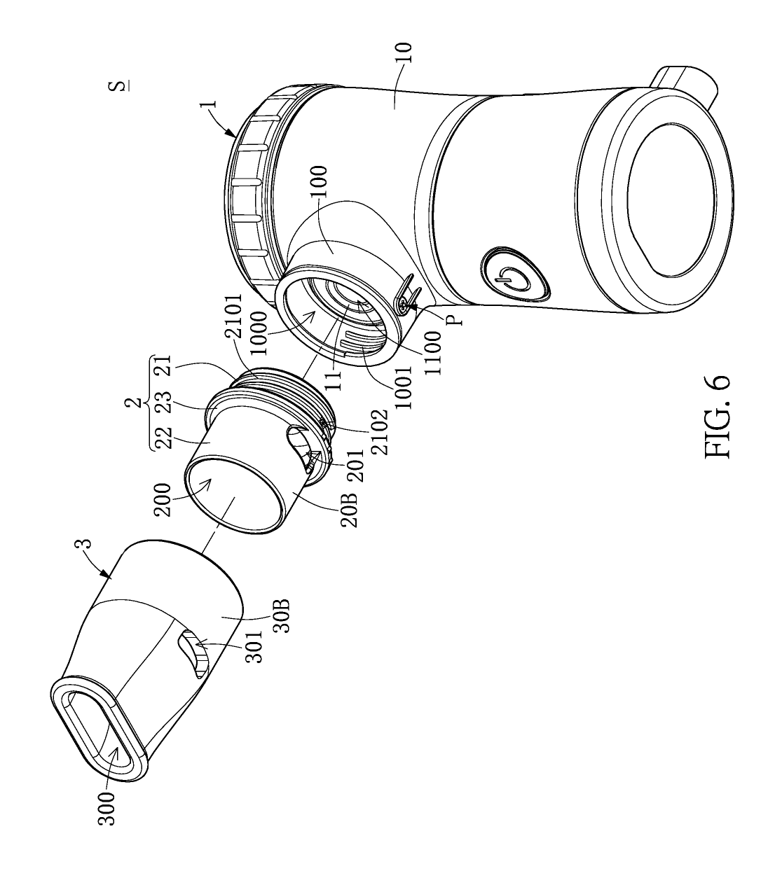

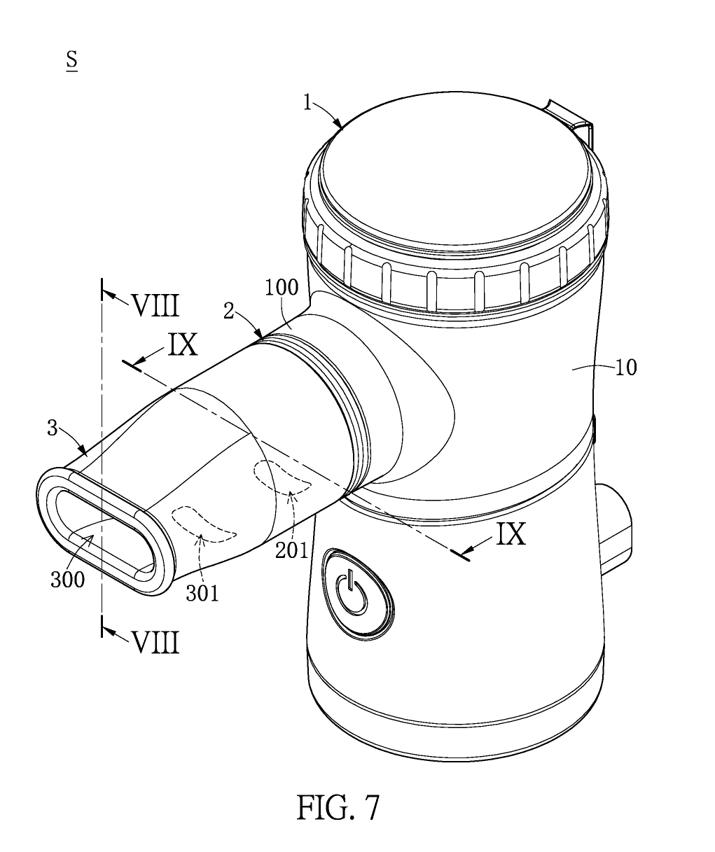

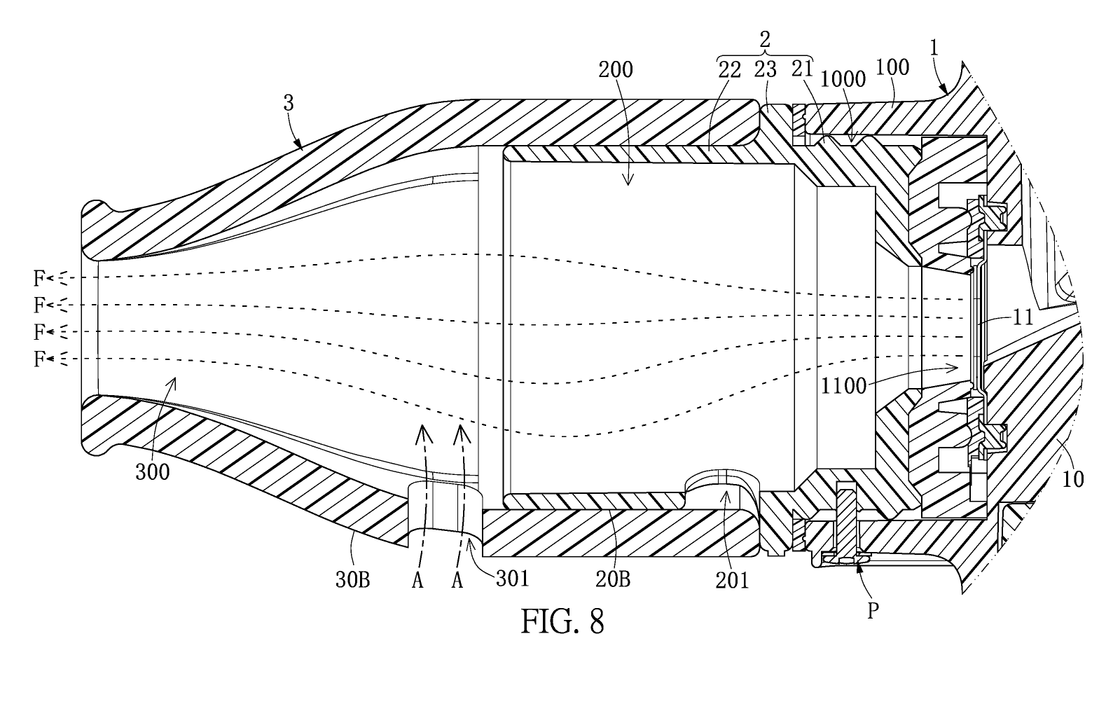

Referring to FIG. 6 to FIG. 9, a second embodiment of the present disclosure provides a nebulizer assembly S including a nebulizer host 1 and a main flow-guiding element 2. Through comparison between FIG. 6 and FIG. 1, FIG. 7 and FIG. 2, and FIG. 8 and FIG. 3, it can be seen that the second embodiment of the present disclosure differs from the first embodiment in that, among other aspects, the nebulizer assembly S of the second embodiment further includes an auxiliary flow-guiding element 3 (or namely, a “second flow-guiding element,” that is, the term “auxiliary” is interchangeable with the term “second” regarding the auxiliary flow-guiding element throughout this disclosure), and that the auxiliary flow-guiding element 3 is disposed on the main flow-guiding element 2, so that the second matching portion 22 is embedded within the auxiliary flow-guiding element 3. That is, in certain embodiments, the second matching portion is configured to be embedded within the auxiliary flow-guiding element. In addition, the auxiliary flow-guiding element 3 has an auxiliary flow-guiding channel 300 and at least one auxiliary bottom flow-guiding opening 301 communicating with the auxiliary flow-guiding channel 300. The auxiliary bottom flow-guiding opening 301 penetrates a bottom portion 30B of the auxiliary flow-guiding element 3. In certain embodiments, the auxiliary flow-guiding element 3 may be a mouthpiece for a user to hold in the mouth.

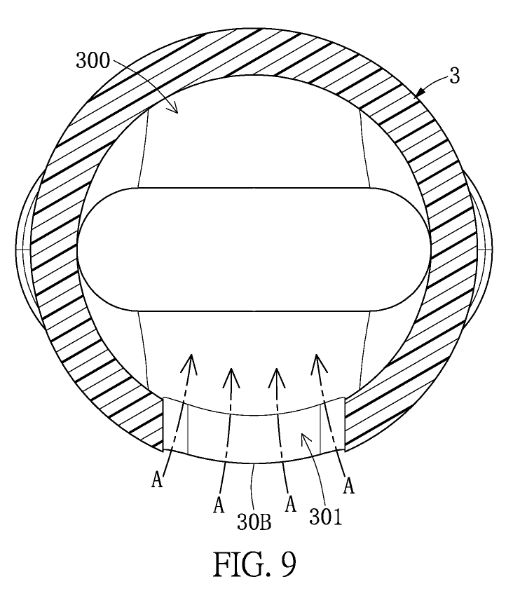

Further, referring to FIG. 8 and FIG. 9, when the nebulizer host 1 is turned on, a nebulized fluid F generated by the nebulizing module 11 of the nebulizer host 1 can be sent to the auxiliary flow-guiding channel 300 of the auxiliary flow-guiding element 3 through the guiding of the main flow-guiding element 2. That is, the main flow-guiding element 2 is also configured to guide the nebulized fluid F to the auxiliary flow-guiding channel 300 of the auxiliary flow-guiding element 3. When the nebulized fluid F is sent to the auxiliary flow-guiding channel 300 of the auxiliary flow-guiding element 3, an outside air A can be introduced into the auxiliary flow-guiding channel 300 through the auxiliary bottom flow-guiding opening 301. As a result, as shown in FIG. 8, the nebulized fluid F in the auxiliary flow-guiding channel 300 can be propelled towards a direction away from the at least one auxiliary bottom flow-guiding opening 301 by the introduction of the outside air A from the auxiliary bottom flow-guiding opening 301. Also, the nebulized fluid F in the auxiliary flow-guiding channel 300 can be propelled along a direction from the main flow-guiding channel 200 to the auxiliary flow-guiding channel 300 by the introduction of the outside air A from the auxiliary bottom flow-guiding opening 301. That is, when the nebulized fluid F generated by the nebulizing module 11 is sent to the auxiliary flow-guiding channel 300 of the auxiliary flow-guiding element 3 by the guiding of the main flow-guiding element 2, a negative pressure is formed in the auxiliary flow-guiding channel 300, enabling the outside air A to be introduced into the auxiliary flow-guiding channel 300 through the auxiliary bottom flow-guiding opening 301. Therefore, the nebulized fluid F in the auxiliary flow-guiding channel 200 can be propelled towards a direction away from the at least one auxiliary bottom flow-guiding opening 301 and along a direction from the main flow-guiding channel 200 to the auxiliary flow-guiding channel 300 by the introduction of the outside air A from the auxiliary bottom flow-guiding opening 301.

For example, when the nebulized fluid F carries a plurality of therapeutic particles, since the nebulized fluid F in the auxiliary flow-guiding channel 300 can be propelled towards a direction away from the at least one auxiliary bottom flow-guiding opening 301 by the introduction of the outside air A, the nebulized fluid F carrying the therapeutic particles will not easily fall (attach) onto the inner surface of the main flow-guiding element 2 and the auxiliary flow-guiding element 3, so that medicine waste can be reduced. In this way, by employing the auxiliary flow-guiding element 3 with the auxiliary bottom flow-guiding opening 301, the present disclosure enables the therapeutic particles carried by the nebulized fluid F to be smoothly carried to the outside of the auxiliary flow-guiding element 3, so as to provide a sufficiently effective dosage of medicine for a user to inhale (that is, improving the effectiveness of medicating). However, the present disclosure is not limited to this example.

In summary, with reference to FIG. 6 to FIG. 8, a second embodiment of the present disclosure provides a nebulizer assembly S including a nebulizer host 1, a main flow-guiding element 2 and an auxiliary flow-guiding element 3. The main flow-guiding element 2 is disposed on the nebulizer host 1. The main flow-guiding element 2 has a main flow-guiding channel 200 and at least one main bottom flow-guiding opening 201 communicating with the main flow-guiding channel 200. The main bottom flow-guiding opening 201 penetrates a bottom portion 20B of the main flow-guiding element 2. As a result, a nebulized fluid F generated by the nebulizer host 1 can be sent to the main flow-guiding channel 200 of the main flow-guiding element 2, and an outside air A can be introduced through the main bottom flow-guiding opening 201 into the main flow-guiding channel 200. Further, the auxiliary flow-guiding element 3 is disposed on the main flow-guiding element 2. The auxiliary flow-guiding element 3 has an auxiliary flow-guiding channel 300 and at least one auxiliary bottom flow-guiding opening 301 communicating with the auxiliary flow-guiding channel 300. The auxiliary bottom flow-guiding opening 301 penetrates a bottom portion 30B of the auxiliary flow-guiding element 3. As a result, the nebulized fluid F generated by the nebulizer host 1 can be sent to the auxiliary flow-guiding channel 300 of the auxiliary flow-guiding element 3 through the guiding of the main flow-guiding element 2, and the outside air A can be introduced through the auxiliary bottom flow-guiding opening 301 into the auxiliary flow-guiding channel 300.

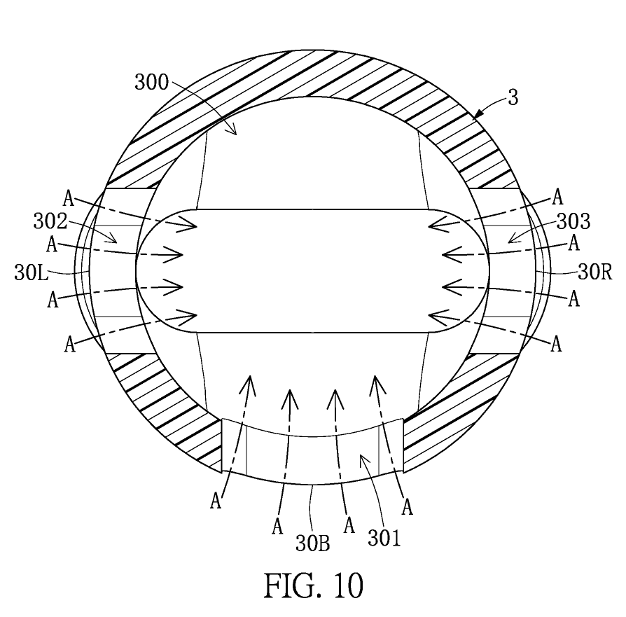

It should be noted that, as shown in FIG. 10, the auxiliary flow-guiding element 3 further has at least one auxiliary left flow-guiding opening 302 communicating with the auxiliary flow-guiding channel 300, and at least one auxiliary right flow-guiding opening 303 communicating with the auxiliary flow-guiding channel 300. In certain embodiments, the auxiliary left flow-guiding opening 302 penetrates a left end portion 30L of the auxiliary flow-guiding element 3, and the auxiliary right flow-guiding opening 303 penetrates a right end portion 30R of the auxiliary flow-guiding element 3. In this way, the outside air A can be introduced into the auxiliary flow-guiding channel 300 through the auxiliary left flow-guiding opening 302 and the auxiliary right flow-guiding opening 303. In certain embodiments, when the nebulized fluid F generated by the nebulizing module 11 is sent to the auxiliary flow-guiding channel 300 of the auxiliary flow-guiding element 3, the outside air A is introduced into the auxiliary flow-guiding channel 300 by passing through the at least one auxiliary left flow-guiding opening 302 and the at least one auxiliary right flow-guiding opening 303. When the outside air A is introduced into the auxiliary flow-guiding channel 300 through the auxiliary left flow-guiding opening 302 and the auxiliary right flow-guiding opening 303, the nebulized fluid F inside the auxiliary flow-guiding channel 300 can be propelled along a direction from the main flow-guiding channel 200 to the auxiliary flow-guiding channel 300 by the introduction of the outside air A through the auxiliary left flow-guiding opening 302 and the auxiliary right flow-guiding opening 303. In certain embodiments, the auxiliary left flow-guiding opening 302 and the auxiliary right flow-guiding opening 303 are relatively close to the auxiliary bottom flow-guiding opening 301. For example, at least one of the centers of the auxiliary left flow-guiding opening 302 and the auxiliary right flow-guiding opening 303 is closer to a center of the auxiliary bottom flow-guiding opening 301 than to a center of a vertical projection of the auxiliary bottom flow-guiding opening 301 projected along an axis perpendicular to a plane where the auxiliary bottom flow-guiding opening 301 is located and projected on a side of the auxiliary flow-guiding element 3 opposite to the auxiliary bottom flow-guiding opening 301, allowing the nebulized fluid F in the auxiliary flow-guiding channel 300 to be propelled towards a direction away from the at least one auxiliary bottom flow-guiding opening 301 to a greater extent by the introduction of the outside air A through the auxiliary left flow-guiding opening 302 and the auxiliary right flow-guiding opening 303.

It is worth mentioning that, in the second embodiment, the main flow-guiding channel 200 may not have the main bottom flow-guiding opening 201. That is, depending on different usage requirements, the main bottom flow-guiding opening 201 may be formed at the bottom portion 20B of the main flow-guiding element 2, or be omitted.

Accordingly, the second embodiment of the present disclosure provides a nebulizer assembly S including a nebulizer host 1, a main flow-guiding element 2 and an auxiliary flow-guiding element 3. The main flow-guiding element 2 is disposed on the nebulizer host 1. The auxiliary flow-guiding element 3 is disposed on the main flow-guiding element 2. The auxiliary flow-guiding element 3 has an auxiliary flow-guiding channel 300 and at least one auxiliary bottom flow-guiding opening 301 communicating with the auxiliary flow-guiding channel 300. The auxiliary bottom flow-guiding opening 301 penetrates a bottom portion 30B of the auxiliary flow-guiding element 3. As a result, the nebulized fluid F generated by the nebulizer host 1 can be sent to the auxiliary flow-guiding channel 300 of the auxiliary flow-guiding element 3 through the guiding of the main flow-guiding element 2, and the outside air A can be introduced through the auxiliary bottom flow-guiding opening 301 into the auxiliary flow-guiding channel 300.

### Third Embodiment

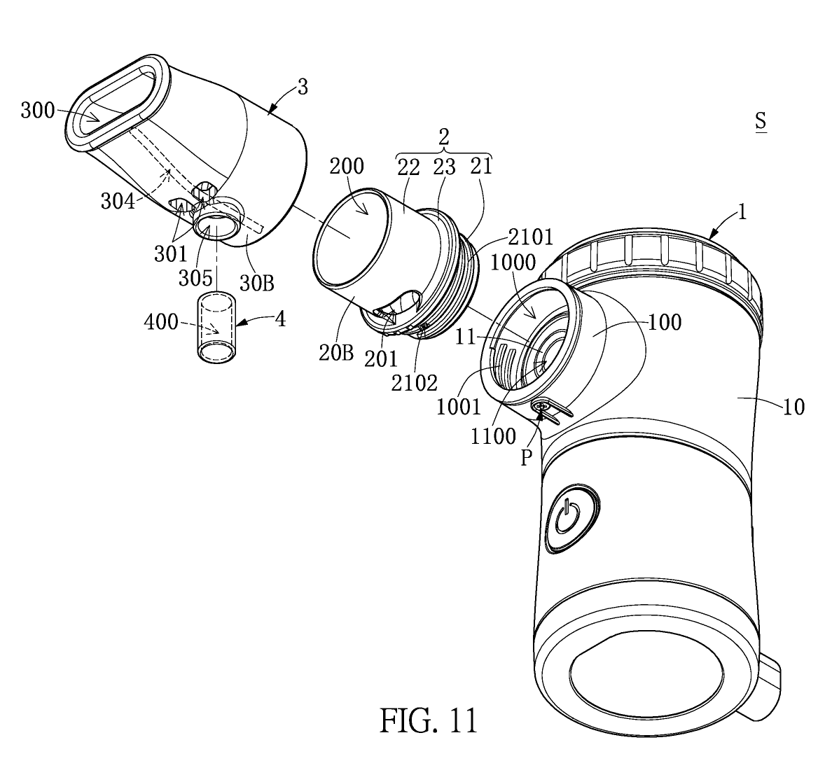

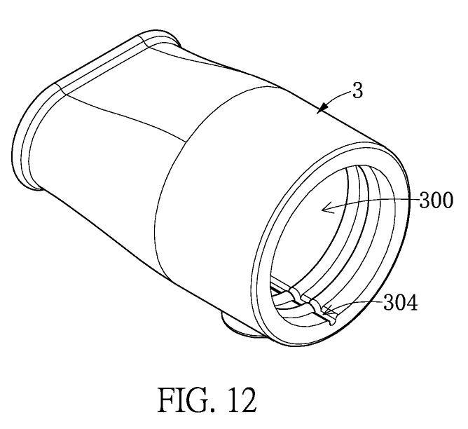

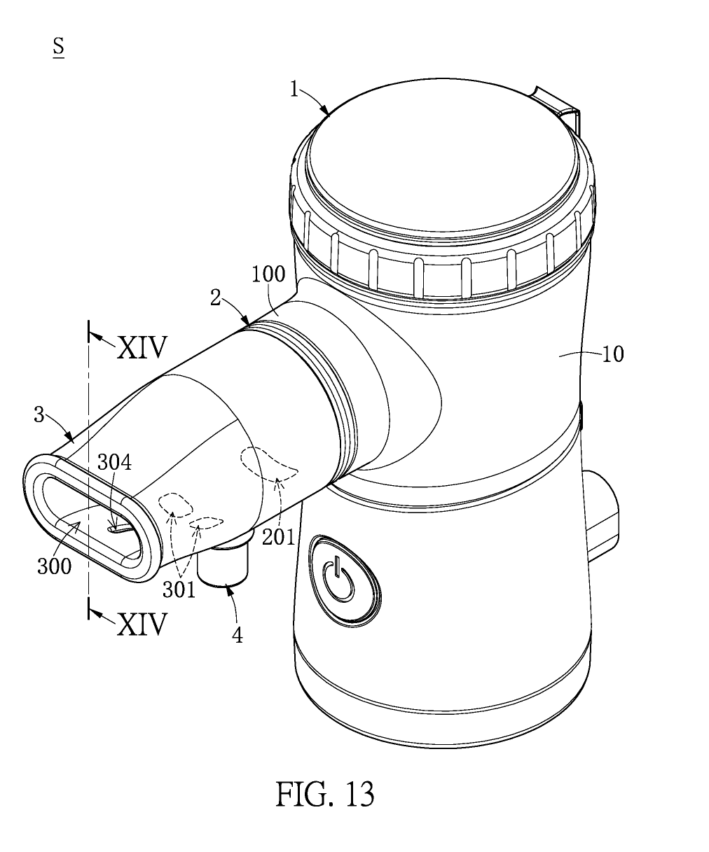

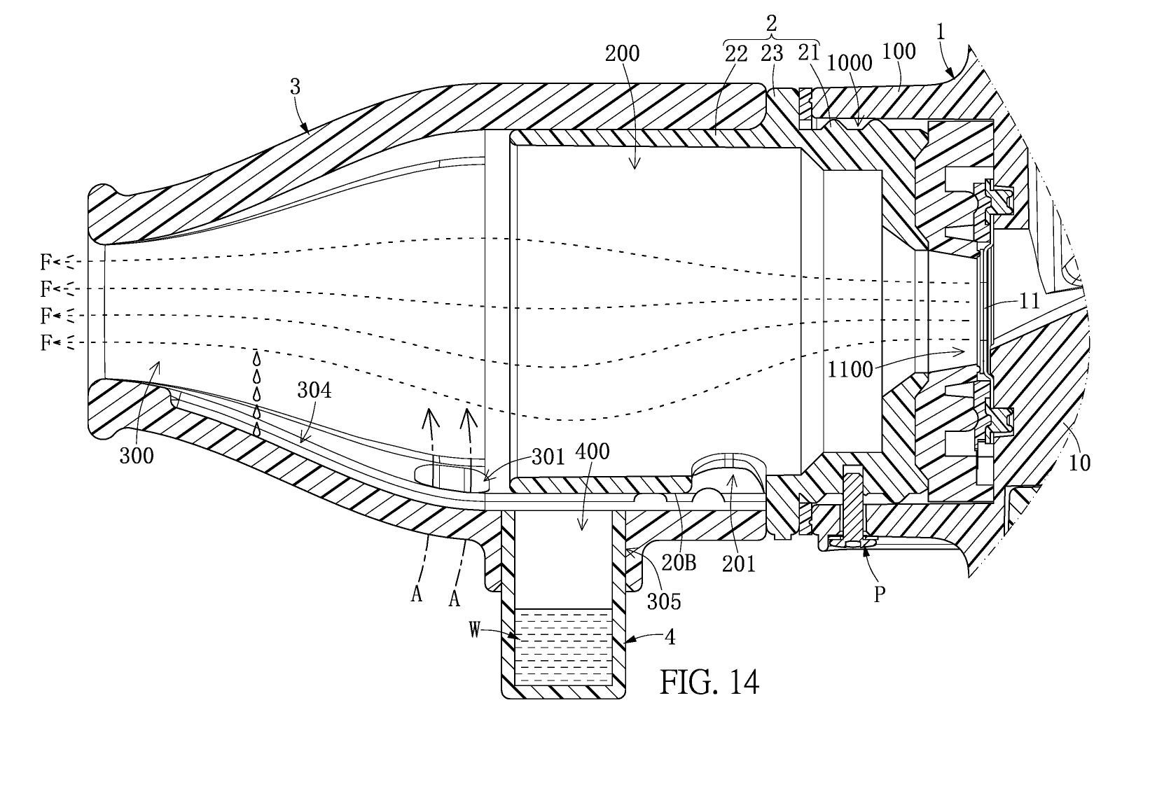

Referring to FIG. 11 to FIG. 14, a third embodiment of the present disclosure provides a nebulizer assembly S including a nebulizer host 1, a main flow-guiding element 2 and an auxiliary flow-guiding element 3. Through comparison between FIG. 11 and FIG. 6, FIG. 13 and FIG. 7, and FIG. 14 and FIG. 8, it can be seen that the third embodiment of the present disclosure differs from the second embodiment in that, among others, the nebulizer assembly S of the third embodiment further includes a fluid collector 4 detachably disposed on the bottom portion 30B of the auxiliary flow-guiding element 3, and the fluid collector 4 has a fluid collection container 400. In addition, the two auxiliary bottom flow-guiding openings 301 shown in FIGS. 11, 13 and 14 for the third embodiment are for illustration purpose only, as the number of the auxiliary bottom flow-guiding opening 301 may vary depending on different usage requirements.

In certain embodiments, the inner surface of the auxiliary flow-guiding element 3 is formed with a fluid-guiding groove 304, and the fluid-guiding groove 304 is adjacent to the bottom portion 30B of the auxiliary flow-guiding element 3. In certain embodiments, the fluid-guiding groove 304 is formed at a lowest bottom portion of the inner surface of the auxiliary flow-guiding element 3. However, the present disclosure is not limited thereto. In addition, the auxiliary flow-guiding element 3 includes a fluid-communicating hole 305 communicating between the fluid-guiding groove 304 and the fluid collection container 400. As a result, the fluid W collected by the fluid-guiding groove 304 flows towards the fluid-communicating hole 305 by the guiding of the fluid-guiding groove 304 and is collected in the fluid collection container 400. That is, the fluid-guiding groove is configured to guide the fluid W (for example, water droplets) formed from partial liquidation of the nebulized fluid F to flow towards the fluid-communicating hole 305, so that the fluid W can be collected in the fluid collection container 400.

### Fourth Embodiment

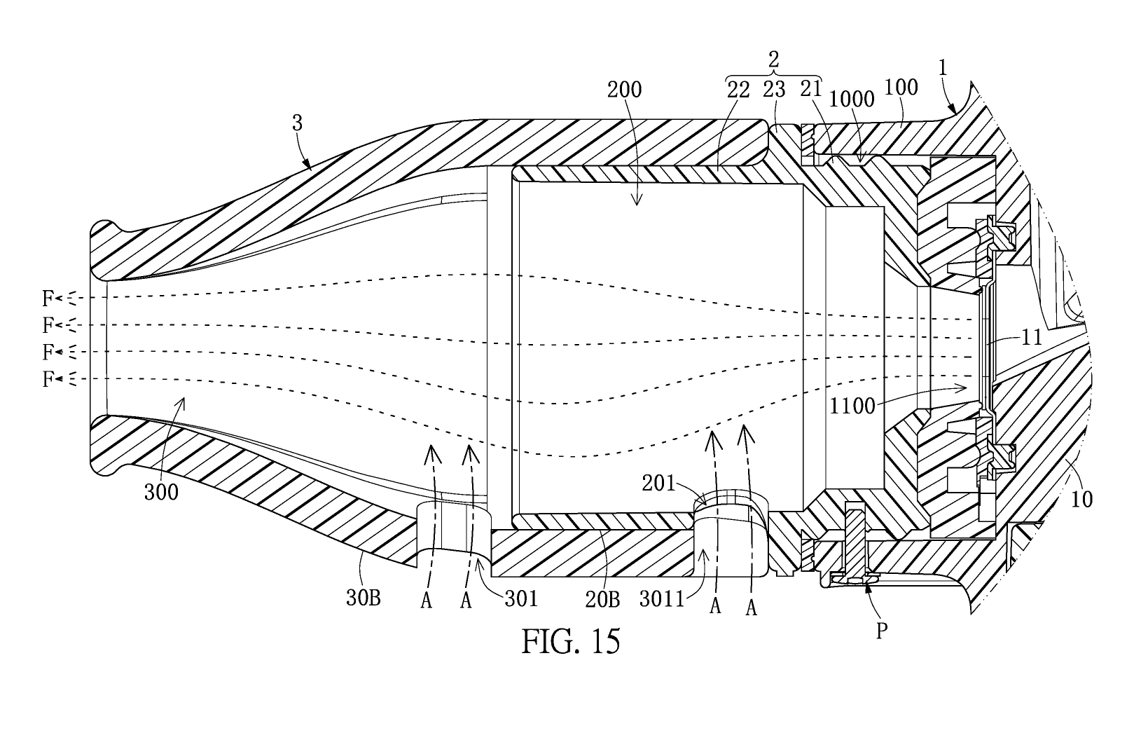

Referring to FIG. 15, a fourth embodiment of the present disclosure provides a nebulizer assembly S including a nebulizer host 1, a main flow-guiding element 2 and an auxiliary flow-guiding element 3. Through comparison between FIG. 15 and FIG. 8, it can be seen that the fourth embodiment of the present disclosure differs from the second embodiment in that, among other aspects, the auxiliary flow-guiding element 3 of the fourth embodiment further includes another auxiliary bottom flow-guiding opening 3011, which corresponds to and communicates with the main bottom flow-guiding opening 201, so that an outside air A can pass through the auxiliary bottom flow-guiding opening 3011 and the main bottom flow-guiding opening 201 in sequence and be introduced into the main flow-guiding channel 200. As a result, the nebulized fluid F can be propelled towards a direction away from the main bottom flow-guiding opening 201 and the auxiliary bottom flow-guiding opening 3011 by the introduction of the outside air A through the auxiliary bottom flow-guiding opening 3011 and the main bottom flow-guiding opening 201.

### Fifth Embodiment

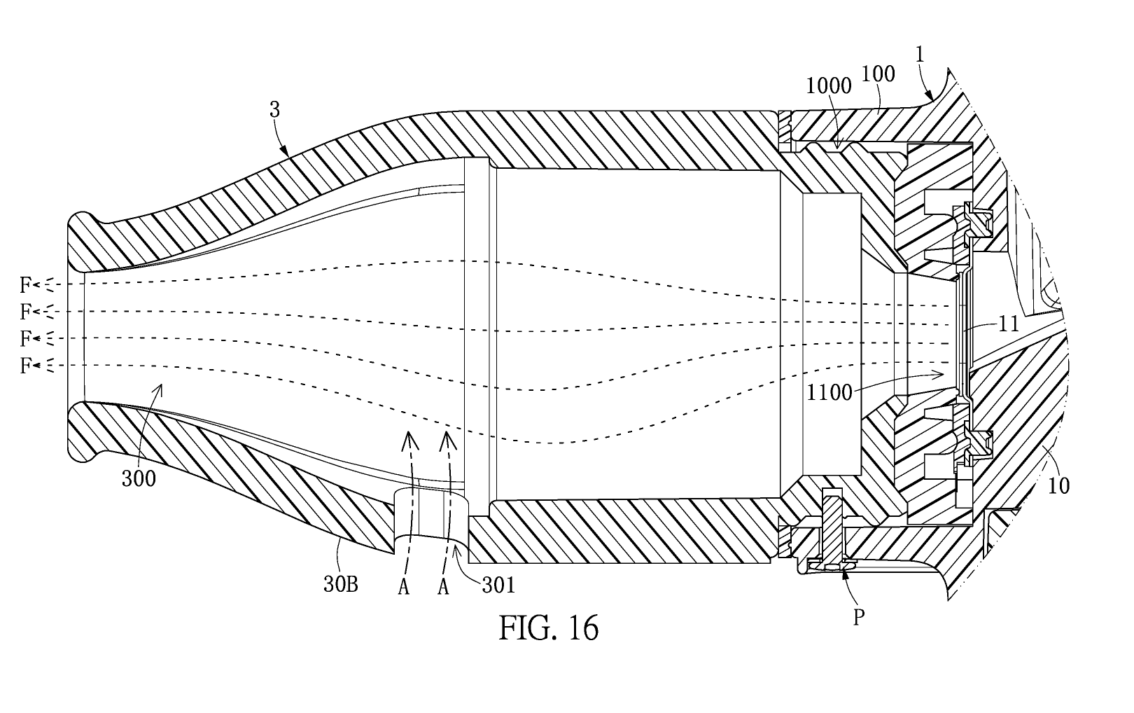

Referring to FIG. 16, a fifth embodiment of the present disclosure provides a nebulizer assembly S including a nebulizer host 1 and an auxiliary flow-guiding element 3. Through comparison between FIG. 16 and FIG. 8, it can be seen that the fifth embodiment of the present disclosure differs from the second embodiment in that, among other aspects, the auxiliary flow-guiding element 3 of the fifth embodiment is directly disposed on the nebulizer host 1 without involving the main flow-guiding element 2.

In certain embodiments, the auxiliary flow-guiding element 3 has an auxiliary flow-guiding channel 300 and at least one auxiliary bottom flow-guiding opening 301 communicating with the auxiliary flow-guiding channel 300. The auxiliary bottom flow-guiding opening 301 penetrates a bottom portion 30B of the auxiliary flow-guiding element 3. As a result, a nebulized fluid F generated by the nebulizer host 1 can be sent to the auxiliary flow-guiding channel 300 of the auxiliary flow-guiding element 3, and an outside air A can be introduced through the auxiliary bottom flow-guiding opening 301 into the auxiliary flow-guiding channel 300.

That is, when the nebulized fluid F generated by the nebulizing module 11 is sent to the auxiliary flow-guiding channel 300 of the auxiliary flow-guiding element 3, a negative pressure is formed in the auxiliary flow-guiding channel 300, enabling the outside air A to be introduced into the auxiliary flow-guiding channel 300 through the auxiliary bottom flow-guiding channel 301. Therefore, the nebulized fluid F in the auxiliary flow-guiding channel 300 can be propelled towards a direction away from the at least one auxiliary bottom flow-guiding opening 301 and along a direction from the main flow-guiding channel 200 to the auxiliary flow-guiding channel 300 by the introduction of the outside air A from the auxiliary bottom flow-guiding channel 301.

For example, when the nebulized fluid F carries a plurality of therapeutic particles, since the nebulized fluid F in the auxiliary flow-guiding channel 300 can be propelled towards a direction away from the auxiliary bottom flow-guiding opening 301 by the introduction of the outside air A, the nebulized fluid F carrying the therapeutic particles will not easily fall (attach) onto the inner surface of auxiliary flow-guiding element 3, so that medicine waste can be reduced. In this way, by employing the auxiliary flow-guiding element 3 with the auxiliary bottom flow-guiding opening 301, the present disclosure enables the therapeutic particles carried by the nebulized fluid F to be smoothly carried to the outside of the auxiliary flow-guiding element 3, so as to provide a sufficiently effective dosage of medicine for a user to inhale (that is, improving the effectiveness of medicating). However, the present disclosure is not limited to this example.

### Sixth Embodiment

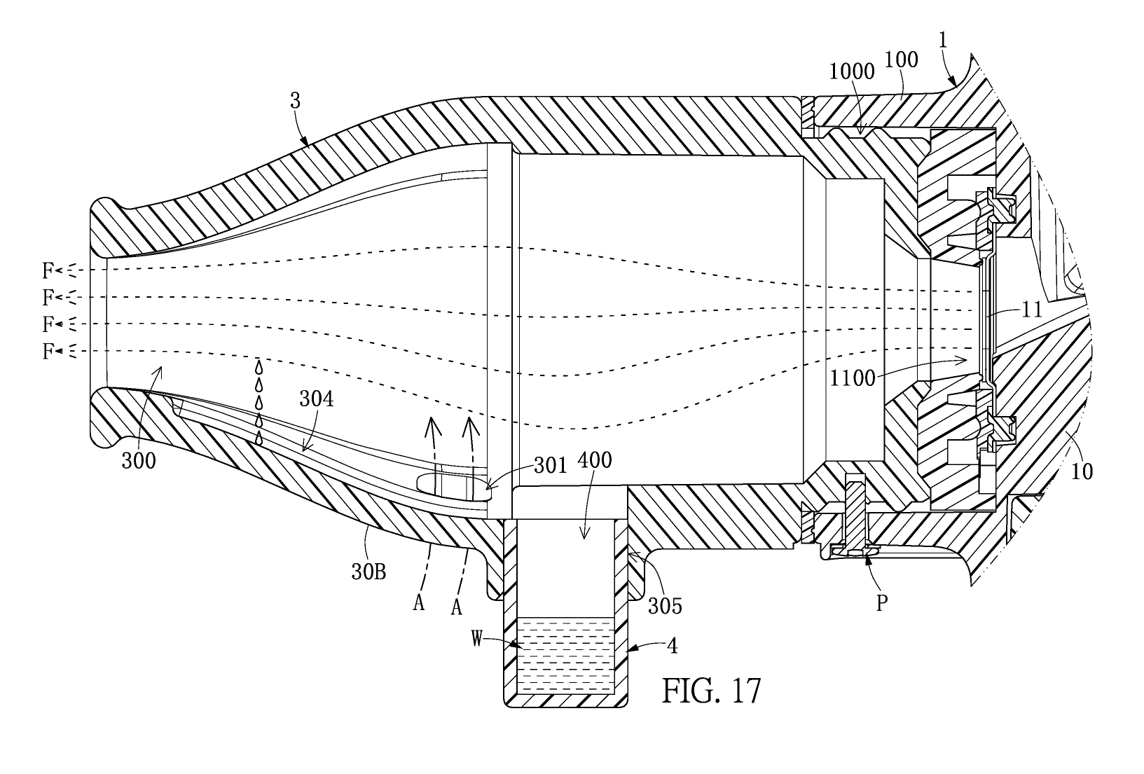

Referring to FIG. 17, a sixth embodiment of the present disclosure provides a nebulizer assembly S including a nebulizer host 1 and an auxiliary flow-guiding element 3. Through comparison between FIG. 17 and FIG. 16, it can be seen that the sixth embodiment of the present disclosure differs from the fifth embodiment in that, among other aspects, the nebulizer assembly S of the sixth embodiment further includes a fluid collector 4.

In certain embodiments, the fluid collector 4 is detachably disposed on the bottom portion 30B of the auxiliary flow-guiding element 3, and the fluid collector 4 has a fluid collection container 400. In addition, the inner surface of the auxiliary flow-guiding element 3 is formed with a fluid-guiding groove 304, and the fluid-guiding groove 304 is adjacent to the bottom portion 30B of the auxiliary flow-guiding element 3. In certain embodiments, the fluid-guiding groove 304 is formed on a lowest bottom portion of the inner surface of the auxiliary flow-guiding element 3. However, the present disclosure is not limited thereto. In addition, the auxiliary flow-guiding element 3 includes a fluid-communicating hole 305 communicating between the fluid-guiding groove 304 and the fluid collection container 400. As a result, the fluid W collected by the fluid-guiding groove 304 flows towards the fluid-communicating hole 305 by the guiding of the fluid-guiding groove 304 and is collected in the fluid collection container 400. That is, the fluid-guiding groove is configured to guide the fluid W (for example, water droplets) formed from partial liquidation of the nebulized fluid F to flow towards the fluid-communicating hole 305, so that the fluid W can be collected in the fluid collection container 400.

It is worth noting that the auxiliary flow-guiding element 3 can be a spray nozzle or a mouthpiece. That is, the fluid-guiding groove 304 is formed on a lowest bottom portion of the inner surface of a spray nozzle or a mouthpiece, and the fluid collector 4 with the fluid collection container 400 can be detachably disposed at a bottom portion of a spray nozzle or a mouthpiece.

One of the beneficial effects of the present disclosure is that, through technical features of the auxiliary flow-guiding element 3 having an auxiliary flow-guiding channel 300 and at least one auxiliary bottom flow-guiding opening 301, and that of the at least one auxiliary bottom flow-guiding opening 301 penetrating a bottom portion 30B of the auxiliary flow-guiding element 3, the nebulizer assembly S and the auxiliary flow-guiding element 3 of the present application enable a nebulized fluid F generated by the nebulizer host 1 to be sent into the auxiliary flow-guiding channel 300 of the auxiliary flow-guiding element 3, and enable an outside air A to be introduced into the auxiliary flow-guiding channel 300 through the auxiliary bottom flow-guiding opening 301. As a result, the nebulized fluid F in the auxiliary flow-guiding channel 300 can be propelled towards a direction away from the at least one auxiliary bottom flow-guiding opening 301 by the introduction of the outside air A.

That is, when the nebulized fluid F generated by the nebulizing module 11 is sent to the auxiliary flow-guiding channel 300 of the auxiliary flow-guiding element 3, a negative pressure is formed in the auxiliary flow-guiding channel 300, enabling the outside air A to be introduced into the auxiliary flow-guiding channel 300 through the auxiliary bottom flow-guiding opening 301. Therefore, the nebulized fluid F in the auxiliary flow-guiding channel 300 can be propelled towards a direction away from the at least one auxiliary bottom flow-guiding opening 301 and along a direction from the main flow-guiding channel 200 to the auxiliary flow-guiding channel 300 by the introduction of the outside air A from the auxiliary bottom flow-guiding opening 301.

For example, when the nebulized fluid F carries a plurality of therapeutic particles, since the nebulized fluid F in the auxiliary flow-guiding channel 300 can be propelled towards a direction away from the at least one auxiliary bottom flow-guiding opening 301 by the introduction of the outside air A, the nebulized fluid F carrying the therapeutic particles will not easily fall (attach) onto the inner surface of the auxiliary flow-guiding element 3, so that medicine waste can be reduced. In this way, by employing the auxiliary flow-guiding element 3 with the auxiliary flow-guiding channel 301, the present disclosure enables the therapeutic particles carried by the nebulized fluid F to be smoothly carried to the outside of the auxiliary flow-guiding element 3, so as to provide a sufficiently effective dosage of medicine for a user to inhale (that is, improving the effectiveness of medicating). However, the present disclosure is not limited to this example.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nebulizer assembly, comprising:
a nebulizer host including:
a casing module including a communicating tube having an outlet channel; and
a nebulizing module disposed in the casing module and having an outlet communicating with the outlet channel;
a main flow-guiding element disposed on the casing module and corresponding to the nebulizing module; and
an auxiliary flow-guiding element disposed on the main flow-guiding element and including:
an auxiliary flow-guiding channel; and
at least one auxiliary bottom flow-guiding opening, communicating with the auxiliary flow-guiding channel and penetrating a bottom portion of the auxiliary flow-guiding element,
wherein the nebulizing module of the nebulizer host is configured to generate a nebulized fluid, and the main flow-guiding element is configured to guide the nebulized fluid to the auxiliary flow-guiding channel of the auxiliary flow-guiding element; and
wherein when the nebulized fluid is sent to the auxiliary flow-guiding channel of the auxiliary flow-guiding element through guiding of the main flow-guiding element, an outside air is introduced into the auxiliary flow-guiding channel by passing through the at least one auxiliary bottom flow-guiding opening, and the nebulized fluid in the auxiliary flow-guiding channel is propelled towards a direction away from the at least one auxiliary bottom flow-guiding opening by the outside air introduced into the auxiliary flow-guiding channel through the at least one auxiliary bottom flow-guiding opening.

2. The nebulizer assembly according to claim 1, wherein the main flow-guiding element includes a first matching portion, a second matching portion and a limiting portion connected between the first matching portion and the second matching portion, the main flow-guiding element is fixed in position on the casing module through matching of the first matching portion and the communicating tube, the second matching portion is configured to be embedded within the auxiliary flow-guiding element, and the limiting portion abuts against an outer surface of the communicating tube; and
wherein an outer surface of the first matching portion is formed with an external thread and a positioning hole, an inner surface of the communicating tube is formed with an internal thread matching with the external thread, and a fixing element penetrates the communicating tube and is connected between the communicating tube and the positioning hole.

3. The nebulizer assembly according to claim 1, wherein the main flow-guiding element includes:
a main flow-guiding channel; and
at least one main bottom flow-guiding opening, communicating with the main flow-guiding channel and penetrating a bottom portion of the main flow-guiding element; and
wherein when the nebulized fluid generated by the nebulizing module of the nebulizer host is sent to the main flow-guiding channel of the main flow-guiding element, the outside air is introduced into the main flow-guiding channel by passing through the at least one main bottom flow-guiding opening, and the nebulized fluid in the main flow-guiding channel is propelled towards a direction away from the at least one main bottom flow-guiding opening by the outside air introduced into the main flow-guiding channel through the at least one main bottom flow-guiding opening.

4. The nebulizer assembly according to claim 1, wherein the main flow-guiding element further includes:
at least one main left flow-guiding opening communicating with the main flow-guiding channel; and
at least one main right flow-guiding opening communicating with the main flow-guiding channel,
wherein the at least one main left flow-guiding opening penetrates a left end portion of the main flow-guiding element, and the at least one main right flow-guiding opening penetrates a right end portion of the main flow-guiding element; and
wherein when the nebulized fluid generated by the nebulizing module of the nebulizer host is sent to the main flow-guiding channel of the main flow-guiding element, the outside air is introduced into the main flow-guiding channel by passing through the at least one main left flow-guiding opening and the at least one main right flow-guiding opening, and the nebulized fluid in the main flow-guiding channel is propelled along a direction from the outlet to the main flow-guiding channel by the outside air introduced into the main flow-guiding channel through the at least one main left flow-guiding opening and the at least one main right flow-guiding opening.

5. The nebulizer assembly according to claim 1, wherein the auxiliary flow-guiding element further includes:
at least one auxiliary left flow-guiding opening communicating with the auxiliary flow-guiding channel; and
at least one auxiliary right flow-guiding opening communicating with the auxiliary flow-guiding channel,
wherein the at least one auxiliary left flow-guiding opening penetrates a left end portion of the auxiliary flow-guiding element, and the at least one auxiliary right flow-guiding opening penetrates a right end portion of the auxiliary flow-guiding element; and
wherein when the outside air is introduced into the auxiliary flow-guiding channel by passing through the at least one auxiliary left flow-guiding opening and at least one auxiliary right flow-guiding opening, the nebulized fluid in the auxiliary flow-guiding channel is propelled along a direction from the main flow-guiding channel to the auxiliary flow-guiding channel by the outside air introduced into the auxiliary flow-guiding channel through the at least one auxiliary left flow-guiding opening and at least one auxiliary right flow-guiding opening.

6. The nebulizer assembly according to claim 1, further comprising
a fluid collector, detachably disposed on the bottom portion of the auxiliary flow-guiding element and including:
a fluid collection container,
wherein an inner surface of the auxiliary flow-guiding element is formed with a fluid-guiding groove, the fluid-guiding groove is adjacent to the bottom portion of the auxiliary flow-guiding element, the auxiliary flow-guiding element is formed with a fluid-communicating hole communicating between the fluid-guiding groove and the fluid collection container, and the fluid-guiding groove is configured to guide a fluid collected by the fluid-guiding groove to flow towards the fluid-communicating hole and to be collected in the fluid collection container.

7. A nebulizer assembly, comprising:
a nebulizer host, configured to generate and send a nebulized fluid;
a main flow-guiding element disposed on the casing module; and
an auxiliary flow-guiding element disposed on the main flow-guiding element and including:
an auxiliary flow-guiding channel; and
at least one auxiliary bottom flow-guiding opening, communicating with the auxiliary flow-guiding channel and penetrating a bottom portion of the auxiliary flow-guiding element,
wherein the main flow-guiding element is configured to guide the nebulized fluid to the auxiliary flow-guiding channel of the auxiliary flow-guiding element; and
wherein when the nebulized fluid generated by the nebulizer host is sent to the auxiliary flow-guiding channel of the auxiliary flow-guiding element through guiding of the main flow-guiding element, an outside air is introduced into the auxiliary flow-guiding channel by passing through the at least one auxiliary bottom flow-guiding opening.

8. The nebulizer assembly according to claim 7, wherein the main flow-guiding element includes:
a main flow-guiding channel; and
at least one main bottom flow-guiding opening, communicating with the main flow-guiding channel and penetrating a bottom portion of the main flow-guiding element; and
wherein when the nebulized fluid generated by the nebulizer host is sent to the main flow-guiding channel of the main flow-guiding element, the outside air is introduced into the main flow-guiding channel by passing through the at least one main bottom flow-guiding opening.

9. The nebulizer assembly according to claim 7, further comprising
a fluid collector, detachably disposed on the bottom portion of the auxiliary flow-guiding element and including:
a fluid collection container,
wherein an inner surface of the auxiliary flow-guiding element is formed with a fluid-guiding groove, the fluid-guiding groove is adjacent to the bottom portion of the auxiliary flow-guiding element, the auxiliary flow-guiding element is formed with a fluid-communicating hole communicating between the fluid-guiding groove and the fluid collection container, and the fluid-guiding groove is configured to guide a fluid collected by the fluid-guiding groove to flow towards the fluid-communicating hole and to be collected in the fluid collection container.

10. An auxiliary flow-guiding element, disposed on a nebulizer host and including:
an auxiliary flow-guiding channel; and
at least one auxiliary bottom flow-guiding opening, communicating with the auxiliary flow-guiding channel and penetrating a bottom portion of the auxiliary flow-guiding element,
wherein when a nebulized fluid generated by the nebulizer host is sent to the auxiliary flow-guiding channel of the auxiliary flow-guiding element, an outside air is introduced into the auxiliary flow-guiding channel by passing through the at least one auxiliary bottom flow-guiding opening.
